(19) United States
(12) Patent Application Publication
(10) Pub. No.: US 2004/0009964 A1
(43) Pub. Date: Jan. 15, 2004

(54) Compositions and methods for the treatment of matrix metalloproteinase-related diseases

(75) Inventor: Christopher J. Capelli, Pittsburgh, PA (US)

Correspondence Address: Peter G. Carroll, MEDLEN & CARROLL, LLP, Suite 350, 101 Howard Street, San Francisco, CA, 94105, US

(73) Assignee: BioInterface Technologies, Inc.

(21) Appl. No.: 10/391,054

(22) Filed: Mar. 18, 2003

Related U.S. Application Data
(60) Provisional application No. 60/367,976, filed on Mar. 27, 2002.

(51) Int. Cl. A61K 31/555; A61K 31/28; A61K 33/38

(52) U.S. Cl. 514/184; 514/495; 424/618

(57) ABSTRACT

The present invention relates to the use of water-soluble photo-stable silver thiosulfate ion complexes that are antimicrobially active and inhibit the activity of metalloproteinases. The present invention describes incorporation of these complexes into medical devices and/or wound dressings to prevent or treat symptomology related to microbial infections and/or conditions of excessive tissue destruction. In particular, these complexes may be useful in the treatment of rheumatoid arthritis and/or osteoarthritis in the prevention and treatment of articular bone joint degradation and subsequent reductions in joint swelling and pain.

### FIELD OF INVENTION

This invention relates to wound healing, antimicrobials and diseases involving excessive tissue destruction. Specifically, this invention relates to stable silver-ion complexes having antimicrobial activity that are also direct inhibitors of matrix metalloproteinases. More specifically, this invention relates to stable silver-ion complexes having antimicrobial activity that inhibit metalloproteinases useful in the treatment and prevention of rheumatoid arthritis and osteoarthritis.

### BACKGROUND OF THE INVENTION

Arthritis is a chronic inflammatory disorder characterized by joint pain. The course of the disease is variable, but can be both debilitating and mutilating. According to conservative estimates approximately 50,000,000 individuals are afflicted with arthritis worldwide. Those individuals are not only subjected to life-long disability and pain, but possibly a shortened life expectancy. Despite considerable investigative efforts there is presently no cure for arthritic disorders. The two most common forms of arthritis are rheumatoid arthritis and osteoarthritis.

Established treatments of rheumatoid arthritis are designed to inhibit either final common pathways of inflammation or immunological mediators. Both approaches are non-specific and, therefore, are associated with severe side effects. Corticosteroids have multiple effects on the immune system and other tissues. Their use is complicated by very high incidence of musculoskeletal, metabolic, neurologic and connective tissue side effects, as well as immunosuppression which may lead to life-threatening infections. For this reason, corticosteroids are usually avoided until all other forms of treatment have failed. See generally, R. Million et al., “Long-Term Study of Management of Rheumatoid Arthritis,” Lancet 1: 812 (1984).

Among the experimental therapies, cyclosporin and anti-tissue necrosis factor-alpha antibodies show some promise. However, serious renal toxicity and non-specific immunosuppression limit significantly the utility of cyclosporin. Due to its ubiquitous role in many cellular functions, anti-tissue necrosis factor therapy may not be a safe therapeutic strategy for rheumatoid arthritis. While preliminary results indicate some promise with the anti-tissue necrosis factor approach, development of lupus-like disease has been noticed in some cases. Similarly, penicillamine, while questionably effective, is toxic even at relatively low doses. See W. F. Kean et al., “The Toxicity Pattern Of D-Penicillamine Therapy,” Arthritis and Rheumatism 23:158 (1980).

Cytotoxic and anti-metabolic drugs, such as methotrexate, azathioprine and cyclophosphamide are non-specifically affecting all rapidly dividing cells and therefore are associated with bone marrow and gastrointestinal toxicity and increased incidence of malignancy. In addition, methotrexate treatment of rheumatoid arthritis has been reported to induce liver damage and lung disease which may be fatal. See J. A. Engelbrecht et al., “Methotrexate Pneumonitis After Low-Dose Therapy for Rheumatoid Arthritis,” Arthritis and Rheumatism 26:1275 (1983) and G. W. Cannon et al., “Acute Lung Disease Associated With Low-Dose Pulse Methotrexate Therapy In Patients With Rheumatoid Arthritis,” Arthritis and Rheumatism 26:1269 (1983).

Most non-steroidal anti-inflammatory drugs (NSAIDs) currently used are designed to non-specifically inhibit prostaglandin synthesis. NSAIDs currently in use modify or diminish—but do not arrest—the inflammatory response. Aspirin remains the most commonly used NSAID. Aspirin toxicity takes many forms, including hypersensitivity reactions, deafness, gastrointestinal and renal toxicity. See generally Simon and Mills, “Nonsteroidal Anti-inflammatory Drugs,” N. Eng. J. Med. 302:1179 (1980).

Thus, most current therapies for rheumatoid arthritis are associated with high incidence of serious side effects. Furthermore, although some medications may offer symptomatic relief, in many cases, they do not significantly modify the progression of joint destruction.

What is needed is a treatment for arthritis having few or no side effects that directly inhibits the causative factor of tissue destruction in these disease states. Furthermore, what is needed is a medically safe and effective parenteral administration thereby allowing the treatment of internal disease states characterized by tissue destruction.

### SUMMARY OF THE INVENTION

This invention relates to the use of stable silver-ion complexes in the treatment of diseases involving excessive tissue destruction mediated by matrix metalloproteinases. More specifically, the present invention relates to the treatment of matrix metalloproteinase-related diseases by water soluble silver-ion matrix metalloproteinase inhibitors.

The contemplated silver-ion complexes are water-soluble matrix metalloproteinase inhibitors suitable to treat matrix metalloproteinase internal diseases and/or microbial infections as well as topical diseases caused by matrix metalloproteinase and/or microbial infections. Furthermore, these water-soluble silver-ion complexes provide effective direct matrix metalloproteinase inhibition at clinically safe concentrations.

This invention contemplates embodiments of water soluble photostable silver-ion complexes derived from reacting silver cations from silver halides (preferably silver chloride) with anions from the sodium thiosulfate salts; the molar ratio of the thiosulfate anions to the silver cations is preferably at least 1:1 and more preferably at least 1.3:1. It is desirable that the silver thiosulfate ion complexes are solid and essentially pure; the term “essentially pure” meaning that the silver thiosulfate ion complexes do not contain significant amounts of waste salts or other substances that interfere with their matrix metalloproteinase inhibition activity. It is even more desirable that the silver thiosulfate ion complexes do not require carrier particles (i.e., the complexes are “carrier-free”). More preferably, these water-soluble stable silver ion complexes are those disclosed in U.S. Pat. No. 6,093,414 to Capelli, herein incorporated by reference. Of particular interest are silver-ion complexes that are a) water-soluble, b) photostable; c) antimicrobial; and d) a non-toxic ligand molecule forming the water-soluble silver ion complex that directly inhibit matrix metalloproteinases.

In other embodiments, these water-soluble silver ion complexes can be used alone or in conjunction with other medical therapeutic agents including anti-microbial agents (e.g. antibiotics, antiseptics, etc.), anti-inflammatory agents (e.g. steroids, aspirin, etc.), chelating agents (e.g. EDTA, EGTA, etc.), anti-cancer agents (e.g. taxol, cis-platinum, etc.), anesthetics (e.g. benzocaine, lidocaine, etc.), non-steroidal antiinflammatory agents (e.g., acetylsalicylates, acetaminophen, etc.) as well as other agents.

The present invention contemplates a method for treatment of a disease characterized by tissue destruction comprising: a) providing: i) a subject, exhibiting at least one symptom associated with a metalloproteinase-associated disease; and ii) an aqueous solution comprising an effective amount of a carrier-free water soluble photostable silver thiosulfate complex, wherein said complex directly inhibits a matrix metalloproteinase; and b) administering said solution to said subject under conditions such that at least one symptom is reduced. In a preferred embodiment, the symptoms of tissue destruction are symptoms of excessive tissue destruction, such as those associated with rheumatoid arthritis, oseteoarthritis, septic arthritis and other diseases.

In one embodiment, the present invention contemplates a method of treatment of a disease characterized by rheumatoid arthritis comprising: a) providing: i) a subject, exhibiting at least one symptom associated with rheumatoid arthritis; and ii) an aqueous solution comprising an effective amount of a carrier-free water soluble photostable silver thiosulfate complex, wherein said complex directly inhibits a matrix metalloproteinase; and b) administering said solution to said subject under conditions such that at least one symptom is reduced.

In another embodiment, the present invention contemplates a method of treatment of a disease characterized by osteoarthritis comprising: a) providing: i) a subject, exhibiting at least one symptom associated with osteoarthritis; and ii) an aqueous solution comprising an effective amount of a carrier-free water soluble photostable silver thiosulfate complex, wherein said complex directly inhibits a matrix metalloproteinase; and b) administering said solution to said subject under conditions such that at least one symptom is reduced.

In another embodiment, the present invention contemplates a method of treatment of a disease characterized by internal tissue destruction comprising: a) providing: i) a subject, exhibiting at least one symptom associated with internal tissue destruction; and ii) a sterile and medically safe aqueous solution comprising an effective amount of a carrier-free water soluble photostable silver thiosulfate complex, wherein said complex directly inhibits a matrix metalloproteinase; and b) administering said solution to said subject under conditions such that at least one symptom is reduced.

The present invention also contemplates a method of treating wounds, comprising the steps of a) providing; i) a subject with a wound, and ii) an aqueous solution comprising an effective amount of a carrier-free water soluble photostable suspended silver thiosulfate ion complex in a base, wherein said complex inhibits a matrix metalloproteinase; and b) administering said solution to said subject, thereby reducing the severity of said wound. In a separate embodiment, the base is anhydrous.

The present invention also contemplates a method of producing medical devices, comprising the steps of: a) providing i) a medical device and ii) an aqueous solution comprising an effective amount of a carrier-free water soluble photostable silver thiosulfate ion complex; and b) contacting said medical device with said solution thereby depositing said complex, wherein said deposited complex inhibits a matrix metalloproteinase. By way of illustration, a carrier-free photostable suspended silver thiosulfate ion complex may be contacted with a urinary catheter comprising a polymer where the silver thiosulfate becomes deposited on to the polymer. Following insertion of the urinary catheter into the subject said complex deposited on the urinary catheter inhibits matrix metalloproteinases.

The present invention also contemplates a method for screening water soluble silver ion complexes having matrix metalloproteinase inhibition activity, comprising: a) providing, i) a carrier-free water soluble photostable silver-ion complex comprising a non-toxic ligand, ii) a solution comprising a substrate and a metalloproteinase; b) contacting said complex with said solution; and c) detecting the activity of said metalloproteinase.

Alternatively, the invention contemplates a method for screening water soluble silver ion complexes having matrix metalloproteinase inhibition activity, comprising: a) providing, i) a carrier-free water soluble photostable silver-ion complex comprising a non-toxic ligand, ii) a microbial culture; b) contacting said complex with said culture; and c) measuring a zone of inhibition.

It is not intended that the present invention be limited to any specific method of administration of a water soluble, silver-based complex. In one embodiment, said complex may be administered by intra-articular injection. In another embodiment, said complex may be administered topically as a gel, ointment, foam, or cream either with, or without, gauze or other absorptive matrices. In yet another embodiment, said complex may be administered parenterally in a sterile solution.

It is not intended that the present invention be limited to any particular subject. In one embodiment, said subject is a human. In another embodiment, said subject is an animal (i.e. non-human). In a further embodiment, said subject is a patient, wherein said “patient” is defined as one exhibiting symptoms requiring medical treatment.

In some embodiments, the medical device comprises a foam polymer while in other embodiments the device comprises non-adherent alginate fibers, wherein the foam or alginate is, optionally, anhydrous.

Other, similar, embodiments contemplate the incorporation of silver thiosulfate ion complexes into cosmetics and personal care products thereby providing metalloproteinase inhibition for treatment of diseases and/or conditions involving excessive tissue destruction.

Another embodiment of the present invention contemplates a medical device for delivering an aerosolized mist of a silver-ion complex to a patient using an apparatus and method providing intrapulmonary drug delivery. Such a device is preferably a hand-held, self-contained, portable device. A more prefeffed device contemplates a computer controlled medical inhalator.

### DEFINITIONS

To further facilitate the understanding of the present invention set forth in the disclosure that follows, a number of terms are defined below.

The term “intra-articular”, as used herein, refers to anything situated within, occurring within, or administered by entry into a joint. Specifically, one embodiment of this invention contemplates the insertion of needle into a joint to inject a silver thiosulfate compound. Such joints may comprise, for example, the knee, shoulder, ankle, elbow, knee, finger, hip etc.

The term “sterile”, as used herein, refers to any solution that is free from living organisms and especially microorganisms.

The term “medically safe”, as used herein, refers to any solution, containing organic or inorganic compounds, that when injected into a living organism will not adversely affect any physiological or biochemical systems.

The term “tissue destruction” as used herein, refers to localized areas of tissue showing symptoms of, for example, inflammation, lymphocytic invasion, proteolytic activity etc., or other similar process whether mediated by immune cells, cytokines, or other mechanisms. The term “excessive tissue destruction” refers to conditions refractory to traditional treatments or untreated conditions exhibiting signs of necrosis. Specifically, an embodiment of this invention contemplates tissue destruction that occurs as a result of metalloproteinase activity.

The term “subject,” as used herein, refers to both humans and animals.

The term “patient,” as used herein, refers to a human subject whose care is under the supervision of a physician or who has been institutionalized (e.g. in a hospital).

The term “receptors” refers to structures expressed by cells which bind molecules having a stereospecific configuration.

The term “antagonist” refers to molecules or compounds which inhibit the action of a “native” or “natural” compound. Antagonists may or may not be homologous to these natural compounds in respect to conformation, charge or other characteristics. Thus, antagonists may be recognized by the same or different receptors that are recognized by the natural compound. Thus, a kinase inhibitor is a kinase antagonist.

The term “base,” as used herein, refers to any substance useful for the suspension of the water soluble silver thiosulfate ion complexes of the present invention. In a preferred embodiment, the base is “anhydrous” (e.g. an ointment) and can be used to suspend a medicinal agent for topical administration. Useful anhydrous bases include, but are not limited to, white petrolatum, AQUAPHOR™ (an ointment base comprising petrolatum, mineral oil, ceresin and lanolin alcohol) and polyethylene glycol (PEG) polymers with molecular weights greater than or equal to 600. The preferred anhydrous base is a PEG ointment composition; an ointment made up of PEGs can absorb and associate a small amount of water so that the water is not free to hydrolyze the thiosulfate. It should be noted that some water is tolerable in the final product but, generally speaking, the presence of water will reduce the shelf-life of the composition. For example, an anhydrous base which contains no water and few, if any, hydroxy or acid groups should have a shelf-life of many years, while a base containing small amounts of water (e.g. less than 5%) would have shorter shelf-life (e.g. less than 6 months). If a PEG ointment base has a very small amount of water (e.g. much less than 1%), the silver thiosulfate ion complexes should be stable enough to provide the product with an acceptable shelf-life (e.g. greater than 1 year). In one embodiment, the base is semi-solid.

The term “carrier,” as used herein, refers to a substance, such as an organic oxide, in which a material can be impregnated and then, if necessary, immobilized through drying. For example, U.S. Pat. No. 5,429,819 to Oka et al. describes the impregnation of a porous carrier (e.g. silica gel) with a solution containing thiosulfate complex salt and thiosulfate metal complex salt. In contrast, the term “carrier” does not refer to the mere suspension of materials like silver thiosulfate ion complexes in a base. The term “carrier-free” refers to being without such carrier particles and porous particulate carriers used as carriers for other materials. For example, the compositions of the present invention are “carrier-free” in that they comprise silver thiosulfate ion complexes (in combination with a medicinal agent?) that do not require such a carrier.

The term “therapeutically effective amount” as used herein, is intended to encompass any concentration of silver thiosulfate ion complex sufficient to reduce either antimicrobial activity or metalloproteinase activity with a concomitant reduction of specific symptoms. Specifically, the present invention contemplates concentrations of silver thiosulfate ion complexes from 0.01% to 30% (w/w) and from 0.1% to 3.0% (w/w). The preferred concentration of silver thiosulfate ion complexes is from 0.2% to 1.5% (w/w).

The term “symptoms of rheumatoid arthritis,” as used herein, is intended to encompass any and all symptoms of rheumatoid arthritis. Where a symptom is said to be “reduced” it is indicated that the degree of such symptom (such as the degree of joint pain or the amount of inflammatory cells in the joints) is diminished. The present invention is not limited to any particular quantitative level. Most importantly, the present invention is not limited to the complete elimination of symptoms.

The term “symptoms of osteoarthritis,” as used herein, is intended to encompass any and all symptoms of osteoarthritis. Where a symptom is said to be “reduced” it is indicated that the degree of such symptom (such as the degree of joint pain or the amount of inflammatory cells in the joints) is diminished. The present invention is not limited to any particular quantitative level. Most importantly, the present invention is not limited to the complete elimination of symptoms.

The term “drug,” as used herein, refers to any medicinal substance used in humans or other animals. Encompassed within this definition are compound analogs, naturally occurring, synthetic and recombinant pharmaceuticals. The term “drug” shall also include “respiratory drug”. The term is intended to encompass the presently available pharmaceutically active drugs used therapeutically and the silver-ion complex contemplated by this invention and further encompasses to be developed therapeutically effective drugs which can be administered by the intrapulmonary route.

The term “wound,” as used herein, includes a burn, cut, sore, blister, rash, ulcer or any other lesion or area of disturbed skin, including but not limited to the wounds associated with poor circulation (e.g. advanced diabetes).

The term “wound dressing” includes foam dressings, thin film dressings, burn dressings, surgical dressings, absorptive dressings, gauze, mesh, bandage, sheets or other types of medical devices used to treat wounds.

The term “severity of the wound is reduced”, as used herein, refers to the natural healing process exemplified by observations such as, a lessening of infection, reduced swelling and inflammation in the wound area, partial wound closure, scab formation, accelerated healing etc.

The term “silver thiosulfate ion complexes,” as used herein, refers to the silver-containing material produced by the process of the present invention and incorporated into the compositions of the present invention. More specifically, the silver thiosulfate ion complexes are obtained by adding a silver halide (e.g. silver chloride) to an aqueous solution and then adding a thiosulfate salt (e.g. sodium thiosulfate) to the solution. Though the benefit provided by the complexes of the present invention is not limited by an understanding of the precise nature of the complexes, the chemical formula of the primary silver thiosulfate ion complexes formed when a large excess of thiosulfate salt is used is represented by [Ag(S₂O₃)₃]⁵⁻. By comparison, the chemical formula of the primary silver thiosulfate ion complexes formed when only a small excess of thiosulfate salt is used is represented by [Ag₂(S₂O₃)₃]⁴⁻. The preferred silver thiosulfate ion complexes are those represented by [Ag₂(S₂O₃)₃]⁴⁻. The resulting silver thiosulfate ion complexes are in a relatively pure solid form, and are stable, highly water soluble and antimicrobially active.

The term “essentially anhydrous silver thiosulfate ion complexes,” as used herein, refers to silver thiosulfate ion complexes that may be essentially free of all remnant water, i.e., they may contain a small amount of water (generally less than 5% of the original amount of water present, preferably less than 1%, and most preferably less than 0.1%), provided that the water does not interfere with the antimicrobial function of the complexes.

The terms “topical” and “topically,” as used herein, refer to the surface of the skin and mucosal tissue, in wounds, in the eyes, nose, mouth, anus and vagina.

The term “internal” refers to, but is not limited to, anatomical regions that are not on the surface of the skin and mucosal tissue.

The term “photostable” means that an object or material is resistant to discoloration when exposed to ambient light for a period of at least 72 hours.

The term “medicinal agents,” as used herein, refers to compounds such as antimicrobial agents (e.g. acyclovir, chloramphenicol, chlorhexidine, chlortetracycline, itraconazole, mafenide, metronidazole, mupirocin, nitrofurazone, oxytetracycline, penicillin, and tertacycline), anti-inflammatory agents (e.g. aspirin and steroids such as betamethasone benzoate, betamethasone valerate, desonide, fluocinolone acetonide, halcinonide, hydrocortisone, and metandienone), and anesthetics (e.g. benzocaine, lidocaine, dibucaine, pramoxine hydrochloride and tetracacine). the present invention contemplates compositions comprising silver thiosulfate ion complexes (i.e. water soluble, silver-based matrix metalloproteinase inhibitors) in combination with such medicinal agents.

The term “medical device” as used herein, refers to any object prescribed or recommended for use by a physician to improve the health and well-being of the subject. For example, “medical devices” may include medical implants, wound care devices, body cavity and personal protection devices, and the like. A preferred “medical device” may include intrapulmonary inhalators or nebulizers capable of administering an aqueous solution.

The term “cosmetics” or “personal care products” as used herein, include any compound intended to improve a subject's aesthetic appearance or social acceptability. Examples of cosmetics and/or personal care products include lipsticks and glosses, lip pencils, mascaras, eye liners, eye shadows, moisturizers, liquid and powder makeup foundations, powder and cream blushes, perfumes, colognes, various creams and toners, etc., and assorted applicators like combs, brushes, sponges, and cotton swabs and balls, and examples of personal care products include deodorants, razors, toothbrushes, shaving creams, shampoos, conditioners, various hair treatments like mousses and sprays, toothpastes, mouthwashes, dental flosses and tapes, sunscreens, moisturizers, tampons, sanitary napkins, panty shields, diapers, baby wipes, facial tissues, toilet tissues, etc.

The term “antimicrobially active” as used herein, includes any compound that produces a zone-of-inhibition when placed on both a lawn of S. aureus (ATCC 25923) and a lawn of E. coli (ATCC 25922) in accordance with Example 10.

The term “matrix metalloproteinase inhibitor” or “metalloproteinase inhibitor”, as used herein include any compound that inhibits the in vitro or in vivo activity of a metalloproteinase such that tissue destruction is reduced.

The term “substrate” as used herein, includes any compound or polymer that is susceptible to enzymatic degradation, where the degradation product is detectable.

The term “ligand” as used herein, includes any compound used to form silver-ion complexes. Specifically, it is contemplated that these ligands be “non-toxic”, which means that, when administered to a subject, no detrimental side effects result.

The term “measuring the zone of inhibition”, as used herein, includes any quantitative assessment determined radially from the center of a silver ion complex deposition on an agar plate to a circumferential edge showing microbial growth.

The term “directly inhibits”, as used herein, includes any reduction in enzyme activity resulting from physical contact between any compound and the enzyme. Specifically, it is contemplated that any silver ion complex compound reduces the activity of metalloproteinase by physical contact.

The term “inspiratory flow” shall be interpreted to mean a value of air flow calculated based on the speed of the air passing a given point along with the volume of the air passing that point with the volume calculation being based on integration of the flow rate data and assuming atmospheric pressure and temperature in the range of about 10° C. to about 35° C.

The term “inspiratory flow profile” shall be interpreted to mean data calculated in one or more monitoring events measuring inspiratory flow and cumulative volume, which profile can be used to determine a point within a patient's inspiratory cycle which is optimal for the release of drug to be delivered to a patient. It is emphasized that the optimal point within the inspiratory cycle for the release of drug is not necessarily calculated based on a point within the inspiratory cycle likely to result in the maximum delivery of drug but rather a point in the cycle most likely to result in the delivery of the reproducible amount of drug to the patient at each release of drug, i.e. repeatability of the amount delivered is important, not maximizing the amount delivered.

### DETAILED DESCRIPTION OF THE INVENTION

The present invention relates to the use of water-soluble silver-ion complexes for their antimicrobial activity as well as direct inhibitors of metalloproteinases involved in various diseases. Specifically, the use of stable, purified water-soluble silver-based compositions comprising carrier-free silver thiosulfate ion complexes are disclosed for the treatment and prevention of diseases involving excessive tissue destruction such as, for example, rheumatoid arthritis and osteoarthritis.

Recent studies have shown that silver ion results in reduced matrix metalloproteinase activity. Unlike gold, however, silver ions are highly reactive to numerous salts and biological molecules. This high reactivity is a characteristic that makes silver ions very useful as broad spectrum antimicrobial agents. Those skilled in the art believe that the high reactivity of silver ion mediates an indirect, and secondary, matrix metalloproteinase inhibition. Silver ions are currently believed to prevent increased matrix metalloproteinase production, rather than directly inhibiting matrix metalloproteinase activity.

One form of silver that is currently being used commercially is nanocrystalline silver (See, e.g., U.S. Pat. No. 6,238,686). This antimicrobial silver composition is deposited using vapor disposition techniques over a large surface area. This process results in a sustained release of silver ions sufficient to produce antimicrobial effects. Investigators studying these nanocrystalline silver compositions in wound healing have postulated several potential mechanisms for matrix metalloproteinase inhibition. (Kirsner et al., Wounds, Volume 13, Number 3, May/June 2001, Supplement C). The proposed potential mechanisms were suggested to involve: 1) reduced bacterial proliferation and bacterial protease activity; 2) reduced neutrophil influx and deposition of neutrophil derived proteases; 3) reduced proinflammatory mediators present in chronic wounds; or 4) direct interaction at various sites on the matrix metalloproteinase itself. However, no evidence was presented to demonstrate a direct inhibitory effect of silver ion on matrix metalloproteinase.

The properties that make dissociated silver ions an excellent antimicrobial agent are not ideal for a direct matrix metalloproteinase inhibitor. As stated earlier, dissociated silver ions are highly reactive and in an in vivo environment, bind to a variety of intracellular and extracellular molecules. This non-specific reactivity reduces the overall bioavailability of silver ion to directly effect matrix metalloproteinase. Although Au(I), Cd(II) and Cu(II) bound directly to human neutrophil collagenase and resulted in noncompetitive inhibition, Ag(I) concentrations up to 10 μM did not directly inhibit matrix metalloproteinase. Higher Ag(I) concentrations were not possible because the silver precipitated in the culture media. (Mallya & Van Wart; 1989)

Even if the inhibition of matrix metalloproteinase was limited to an indirect mechanism, the current forms of silver such as nanocrystalline silver, silver sulfadiazine, silver oxide, etc. are less than ideal because they are not soluble in water. The majority of diseases associated with matrix metalloproteinase (i.e., arthritis, cancer, vascular disease, etc.) are internal, therefore, water insoluble forms of silver are not practical.

This invention contemplates the direct inhibition of metalloproteinases that are involved in a large number of disease states and other conditions in human and other animals. These disease states include, but are not limited to the fields of rheumatology, oncology, cardiology, embryology and dermatology.

The metalloproteinases are a family of enzymes containing zinc at the active site facilitating protein substrate hydrolysis. A subfamily of the metalloproteinase family is known as the matrix metalloproteinases because these enzymes are capable of degrading the major components of articular cartilage and basement membranes. However, for the purpose of this document matrix metalloproteinases and metalloproteinases will be used interchangeably. Matrix metalloproteinases contemplated by this invention include, but are not limited to, stromelysin, collagenase, matrilysin and gelatinase.

Rheumatology

Elevated levels of stromelysin and collagenase have been detected in joints of arthritic humans and animals (Hasty et al., Arthr. Rheum., 33: 388-397 (1990); Krane et al., “In The Control of Tissue Damage,” A. B. Glauert (ed.), Elsevier Sci. Publ., Amsterdam, 1988, Ch. 14, pp. 179-195; Blanckaert et al., Clin. Chim. Acta, 185: 73-80 (1989)). It is believed, therefore, that these diseases result in the loss of articular cartilage.

The cause of arthritic conditions has implicated a role for the matrix metalloproteinases and therefore arthritis (in the context of the present invention) is a “metalloproteinase-associated disease”. Therefore, various transition metal ions have been investigated as possible matrix metalloproteinase inhibitors in arthritic conditions. For example, Panagakos et al. studied the effect of gallium nitrate on matrix metalloproteinase activity (Panagakos et al., “The Effect of gallium nitrate on synoviocyte MMP activity,” Biochimie., 82(2): 147-51 (2000)). The study demonstrated that gallium nitrate can inhibit matrix metalloproteinase activity and may be useful as a modulator of inflammation in arthritis.

Matrix metalloproteinases are well known to possess a number of different binding sites for transition metal ions and other organomercurials. These matrix metalloproteinase binding sites take the form of high affinity activation sites and low affinity inhibition sites. Specifically, human neutrophil collagenase, has at least one binding site where binding of Au(I), Cd(II), Hg(II) and Cu(II) cause collagenase inhibition, but binding by Zn(II) resulted in a retention of activity. (Mallya & Van Wart, “Mechanism of Inhibition of Human Neutrophil Collagenase by Gold (I), Chrysotherapeutic Compounds,” J. Biol. Chem., 264(3): 1594-1601 (1989). Inhibition of matrix metalloproteinase activity with transition metal ion chelating agents also produced data consistent with the concept of a metal-dependent ion binding site. (Mookhtiar et al., (1986) Arch. Biochem. Biophys. 246, 645-649).

Observations of a matrix metalloproteinase metal-dependent ion inhibitory binding site led to investigations involving gold compounds for the clinical treatment of arthritis. (Dash, Metal Ions In Biological Systems, 14:179 (1982); and Elder et al., Chem. Rev. 87:1027 (1987)). The therapeutic action of gold compounds to treat arthritis is limited and the mode of action of anti-arthritic gold drugs is largely unknown. However, Au(I) appears to bind to inhibit metalloproteinases in a noncompetitive manner. (Mallya and Van Wart; (1989))

Gold therapy has a high incidence of bone marrow, renal and mucocutaneous toxicity and is associated with nephropathy. (W. Katz et al., “Proteinuria in Gold-Treated Rheumatoid Arthritis,” Ann. Int. Med. 101:176 (1984)). These problems have led to almost complete abandonment of gold treatments in rheumatoid arthritis therapy.

Stromelysin and collagenase are also implicated in the articular cartilage damage associated with septic arthritis; a form of bacterial infection. These joint bacterial infections elicit an overreactive inflammatory response resulting in permanent damage to structural components. Bacterial-induced arthritis in animal models are associated with the appearance of proteolytic activities (Case et al., J. Clin. Invest., 84:1731-1740 (1989); Williams et al., Arthr. Rheum., 33: 533-541 (1990)). The present invention contemplates silver thiosulfate ion complexes having both antibacterial and metalloproteinase inhibition activity that may be useful in reducing the symptoms of arthritis (including but not limited to septic arthritis).

Oncology

Metastatic tumor invasion may be mediated by secreted metalloproteinases such as stromelysin, collagenase, and gelatinase which are overexpressed in certain metastatic tumor cell lines. Therefore, in the context of the present invention, cancer is a “metalloproteinase-associated disease”. While it is not necessary to understand the underlying mechanism(s) of the invention it is believed that the metalloproteinases penetrate the underlying basement membrane layer to allow tumor cell escape from the primary tumor site into the general circulation. Similarly, the escaped tumor cell may adhere to the blood vessel wall, wherein the basement membrane is again penetrated thus allowing the tumor cell to attach to other tissues. Similarly, stromelysin has been implicated in the degradation of structural components of the glomerular basement membrane (GBM) of the kidney, the major function of which is to restrict passage of plasma proteins into the urine (Baricos et al., Biochem. J., 254:609-612 (1988)). GBM degradation increases plasma protein permeability and results in the release of excess protein into the urine (i.e., proteinuria). These data indicate that metalloproteinase activity, including stromelysin, may play an important role in glomerular diseases having increased GBM permeability.

Periodontal diseases such as gingivitis are also characterized by metalloproteinase expression. Both collagenase and stromelysin activities have been isolated from fibroblasts isolated from inflamed gingiva (Uitto et al., J. Periodontal Res., 16: 417-424 (1981)). Enzyme levels have been correlated to the severity of gum disease (Overall et al., J. Periodontal Res., 22: 81-88 (1987)).

Cardiology

Metalloproteinase activity may also be involved in the rupturing of atherosclerotic plaques leading to coronary thrombosis. Therefore, in the context of the present invention, atherosclerosis is a “metalloproteinase-associated disease”. While it is not necessary to understand the underlying mechanism(s) of the invention it is believed that a tearing or rupturing of atherosclerotic plaques is the most common event initiating coronary thrombosis. Proteolytic enzymes or cytokine activity, such as metalloproteinases, may be responsible for atherosclerotic plaque fissuring by destabilizing and degrading the surrounding connective tissue matrix. Such tearing of these plaques can cause an acute thrombolytic event as blood rapidly flows out of the blood vessel. High levels of stromelysin messenger RNA are localized in individual cells from atherosclerotic plaques removed from heart transplant patients (Henney et al., Proc. Natl. Acad. Sci. USA, 88: 8154-8158 (1991)).

Similarly, degenerative aortic disease is associated with the thinning of the medial aortic wall and implicates a role for matrix metalloproteinase activity. Therefore, in the context of the present invention, degenerative aortic disease is a “metalloproteinase-associated disease”. Aneurysms are often associated with atherosclerosis in this tissue. Increased levels of the matrix metalloproteinases have been identified in patients with aortic aneurysms and aortic stenosis (Vine et al., Clin. Sci., 81: 233-239 (1991)). It is contemplated that inhibition of metalloproteinases by one embodiment of the silver thiosulfate ion complex may prevent cardiovascular disease including but not limited to degradation processes that result in coronary thrombosis, aortic degenerative disease, and aneurysms.

Embryology

Expression of metalloproteinases, including stromelysin and collagenase, is observed in unfertilized eggs and zygotes and at further cleavage stages and increased at the blastocyst stage of fetal development and with endoderm differentiation (Brenner et al., Genes & Develop., 3: 848-859 (1989)). By analogy to tumor invasion, a blastocyst may express metalloproteinases in order to penetrate the extracellular matrix of the uterine wall during implantation. In addition, evidence exists that collagenase is important in ovulation processes. Collagenase apparently facilitates penetration of a covering of collagen over the apical region of the follicle, allowing the ovum to escape. There may also be a role for stromelysin activity during ovulation (Too et al., Endocrin. 115: 1043-1050 (1984)).

Dermatology

Proteolytic processes have also been observed in the ulceration of the cornea following alkali burns (Brown et al., Arch. Ophthalmol., 81: 370-373 (1969)). Collagenolytic and stromelysin activity have also been observed in dystrophobic epidermolysis bullosa (Kronberger et al., J. Invest. Dermatol., 79: 208-211 (1982); Sawamura et al., Biochem. Biophys. Res. Commun., 174: 1003-1008 (1991)).

Imbalance of regulation of matrix metalloproteinases are thought to contribute to numerous problems involved with wound healing. Therefore, in the context of the present invention, abnormal wound healing is a “metalloproteinase-associated disease”. These include persistence and chronic wounds such as venous ulcers, diabetic ulcers and decubitis ulcers.

Finally, other conditions or disease states are thought to be contributed by imbalance of regulation of metalloproteinases. These include inflammation, pain, osteoporosis, multiple sclerosis and other autoimmune or inflammatory disorders dependent on the tissue invasion of leukocytes or other activated migrating cells, acute and chronic neurodegenerative disorders including stroke, head trauma, spinal cord injury, Alzheimer's disease, amyotrophic lateral sclerosis, cerebral amyloid angiopathy, AIDS, Parkinson's disease, Huntington's disease, prion diseases, myasthenia gravis, and Duchenne's muscular dystrophy.

This invention contemplates silver-ion compounds that directly inhibit metalloproteinases that are potentially useful for the treatment or prophylaxis of conditions such as the above mentioned diseases that involve tissue destruction from metalloproteinases. Specifically, these compounds are silver thiosulfate ion complexes that may be administered, for example, topically, intra-articularly or parenterally etc. While it is not necessary to understand the underlying mechanism(s) of the invention it is believed that silver thiosulfate ion complexes inhibit metalloproteinases to reduce excessive tissue destruction in specific diseases or conditions. For example, matrix metalloproteinase inhibitors such as the silver thiosulfate ion complex may be useful the treatment of chronic wounds such as venous ulcers, diabetic ulcers and pressure sores and other skin problems such as bums, scars and psoriasis. Other treatments include, but are not limited to, rheumatoid arthritis, osteoarthritis, osteopenias such as osteoporosis, periodontitis, gingivitis, corneal epidermal or gastric ulceration, and tumor metastasis, invasion and growth. Silver thiosulfate silver complex Matrix metalloproteinase inhibitors are also of potential value in the treatment of neuroinflammatory disorders. These conditions include, but are not limited to, myelin degradation, (e.g., multiple sclerosis). Furthermore, silver thiosulfate silver complex matrix metalloproteinase inhibitors may be of benefit for the management of angiogenesis dependent diseases, which include arthritic conditions, solid tumor growth, psoriasis, proliferative retinopathies, neovascular glaucoma, ocular tumors, angiofibromas and hemangiomas.

Non-silver based therapeutic metalloproteinase inhibitors have been suggested as possible therapeutic agents in many disease states. For example, collagenase inhibition by thiol carboxylic acid derivatives is disclosed in U.S. Pat. Nos. 5,109,000; 4,595,700; and 4,371,466. Hydroxamic acids are suggested as collagenase inhibitors in U.S. Pat. No. 4,599,361 and European Pat. App. Pub. No. 0 236 872. Likewise, hydroxamic acid derivatives are disclosed to inhibit metalloproteinases such as collagenase, stromelysin (proteoglycanase), gelatinase and collagenase (IV) that are involved in tissue degradation in U.S. Pat. Nos. 5,304,604, 5,240,958 and 5,310,763.

Other collagenase inhibitors are disclosed in European Pat. App. Pub. Nos. 0 423 943; 0 273 689; 0 322 184; and 0 185 380, and in International Pat. App. Pub. Nos. WO 88/06890 and WO 94/07481.

Current silver-ion therapy approaches do not control for the high reactivity of silver ions to other molecules in the environment, therefore, the silver ions are unavailable to bind to the matrix metalloproteinase. Current approaches do not allow for the administration of silver ions in sufficiently high concentrations to overcome this reduction in bioavailability in order to observe direct matrix metalloproteinase inhibition. The required silver concentrations would certainly be considered unreasonable and medically unacceptable. Therefore, current compositions of administered silver ions have limited bioavailability and a negligible direct inhibitory effect on matrix metalloproteinase. These limitations in the art have resulted in an inaccurate conclusion that silver inhibits matrix metalloproteinase indirectly, most probably through reductions production, such as synthesis and/or release,

The present invention contemplates water-soluble silver ion complexes that consist of silver ions complexed by ligand molecules. Specifically, these silver ion complexes comprise polymers (i.e. polyoxyethylene glycol; PEG) and small molecules (i.e., thiosulfate). These water-soluble silver ion complexes enable the silver ions to retain antimicrobial activity and matrix metalloproteinase binding ability even in environments having a high concentration of other molecules to which silver ions typically bind. While it is not necessary to understand the underlying mechanism(s) it is believed that the formation of these water-soluble silver complexes enable the silver ions to retain active binding prevent precipitation or losing binding capacity.

This unexpected and surprising discovery occurred when water-soluble silver ion complexes were studied as antimicrobial agents. The antimicrobial activity of silver ion is most likely a result of non-specific binding to molecules within microbes, resulting in the microbe's death. The antimicrobial activity of any compound, including silver ion, may be assessed using a zone of inhibition (ZOI) test in accordance with Example 10. The relative efficacy of two different antimicrobial agents may be determined by comparing the relative sizes of the respective ZOIs. The larger the ZOI, the greater the potential antimicrobial activity.

Silver antimicrobial compounds taught in the art have properties that require free ionic silver to observe antimicrobial activity. Unfortunately, these free silver ions are highly reactive to other molecules in the agar media thereby the antimicrobial effect of the silver ions is totally determined by those ions that have not bound to non-microbial surfaces. Therefore, when current silver compounds are tested for microbial activity, for example using the ZOI test, the silver ions must first dissociate from silver compound, diffuse into the agar and then react with the bacteria. This multi-step process confounds the interpretation of the ZOI size in relation to the true antimicrobial efficacy of silver ion. In sum, the high reactivity of the silver ions to agar molecules results in a large percentage of unreactive silver ions. ZOI antimicrobial testing of silver ion compounds is, therefore, limited and careful interpretation is required when comparing different silver compounds that require dissociated free silver ions for antimicrobial efficacy. These different compounds should have equivalent ZOI sizes because of equivalent dissociation rates that result in equivalent free ionic silver concentrations. Different size ZOI using different compounds usually reflects an increased rate of silver ion dissociation from one silver compound compared to the other.

A surprising discovery revealed that water-soluble silver ion complexes contemplated by certain embodiments of this invention produce significantly larger ZOI's when compared to equal concentrations of antimicrobial silver compounds currently known in the art. While it is not necessary to understand the underlying mechanism(s) of an invention it is believed that the water-soluble silver ion complexes are hindered less by the agar media molecules than dissociated silver ions from compounds currently taught in the art. Specifically, it is believed that the water-soluble silver ion complex embodiments contemplated by this invention do not react, precipitate and/or lose their binding activity by interacting with the other molecules in the agar environment. This reduction in agar media molecule hinderance increases the diffusion capability of the water soluble silver ion complexes thus resulting in a larger ZOI. It is also believed that this phenomenon sufficiently increases the concentration of water-soluble silver ion complexes to provide a direct inhibition of metalloproteinases.

Up until now, Ag(I) has not been found to directly inhibit metalloproteinases because the required concentrations resulted in a silver precipitate in the culture media. This invention contemplates water-soluble silver ion complexes are delivered to metalloproteinases at relatively high concentrations with only minimal precipitation. Therefore, the effective silver concentration in the immediate environment surrounding the metalloproteinase is significantly higher than that seen with silver compounds currently used in the art (i.e. silver salts, nanocrystalline silver, etc.). As a result, water-soluble silver ion complexes of this invention is expected to directly bind and inhibit metalloproteinase activity similar to what is seen by Au(I), Cd(II) and Cu(II).

It is preferred that water-soluble silver ion complexes contemplated by this invention are silver ion complexes that are:

a) water-soluble;

b) photostable;

c) antimicrobial; and

d) the ligand molecule (to form the water-soluble silver ion complex) is relatively non-toxic

More preferably are water-soluble silver complexes as detailed in U.S. Pat. No. 6,093,414 to Capelli hereby incorporated by reference.

The utility of water-soluble silver ion complexes contemplated by this invention to inhibit matrix metalloproteinases may be determined by standard assays well known in the art. For example, by using a collagenase activity assay sold by Chemicon (Product Number 287-11/00) or as described in Example 11.

Alternatively, screening for potential water-soluble silver ion complex compounds for matrix metalloproteinase inhibition according to embodiments of this invention may be performed by using the ZOI test as described in Example 10. Water-soluble silver-ion compounds that produce large ZOIs, when compared to non-water soluble silver compounds having equivalent amounts of silver (i.e. silver salts such as silver nitrate, silver chloride, etc.), are considered good potential matrix metalloproteinase inhibitory agents. Conversely, if the water-soluble silver ion complex does not produce a large ZOI, when compared to non-water soluble silver compounds having equivalent amounts of silver (i.e. silver salts such as silver nitrate, silver chloride, etc.), this is predictive as a strong indicator that the agent poorly dissociates silver ion and would be an ineffective matrix metalloproteinase inhibitor.

It is not intended that the present invention be limited to any particular route of administration. Contemplated routes of administration include, but are not limited to, topical, parenteral, intra-articular etc. A preferred embodiment contemplates intra-articular injection of water soluble silver-ion complex matrix metalloproteinase inhibitors. This method is especially useful to treat subjects suffering from rheumatoid arthritis or osteoarthritis.

To perform an intra-articular injection the targeted joint area is palpated and is then marked, e.g., with firm pressure by a ballpoint pen that has the inked portion retracted. This will leave an impression that will last 10 to 30 minutes (the ballpoint pen technique can also be used with soft tissue injection). The area to be aspirated and/or injected should be carefully cleansed with a good antiseptic, such as one of the iodinated compounds (e.g. providone). Then the needle can be inserted through the ballpoint pen impression.

Helpful equipment includes the following items: alcohol sponges; iodinated solution and surgical soap; gauze dressings (2×2); sterile disposable 3-, 10- and 20-ml syringes; 18- and 20-gauge, 1½-inch needles; 20-gauge spinal needles; 25-gauge, ⅝-inch needles; plain test tubes; heparinized tubes; clean microscope slides and coverslips; heparin to add to heparinized tubes if a large amount of inflammatory fluid is to be placed in the tube; fingernail polish to seal wet preparation; chocolate agar plates or Thayer-Martin medium; tryptic soy broth for most bacteria; anaerobic transport medium (replace periodically to keep culture media from becoming outdated); tubes with fluoride for glucose; plastic adhesive bandages; ethyl chloride; hemostat; tourniquet for drawing of simultaneous blood samples; and 1 percent lidocaine.

It is not intended that the present invention be limited to the intra-articular injection of any specific joint. In one embodiment, the compositions of the present invention are injected into one or more of the following joints in the manner described below.

Knee. The knee is the easiest joint to inject. The patient should be in a supine position with the knee fully extended. The puncture mark is made just posterior to the medial portion of the patella, and an 18- to 20-gauge, 1½-inch needle directed slightly posteriorly and slightly inferiorly. The joint space should be entered readily. On occasion thickened synovium or villous projections may occlude the opening of the needle, and it may be necessary to rotate the needle to facilitate aspiration of the knee when using the medial approach. An infrapatellar plica, a vestigal structure that is also called the ligamentum mucosum, may prevent adequate aspiration of the knee when the medial approach is used. However, the plica should not adversely affect injections or aspirations from the lateral aspect.

Shoulder. Injections in the shoulder are most easily accomplished with the patient sitting and the shoulder externally rotated. A mark is made just medial to the head of the humerus and slightly inferiorly and laterally to the coracoid process. A 20- to 22-gauge, 1½-inch needle is directed posteriorly and slightly superiorly and laterally. One should be able to feel the needle enter the joint space. If bone is hit, the operator should pull back and redirect the needle at a slightly different angle.

The acromioclavicular joint may be palpated as a groove at the lateral end of the clavicle just medial to the shoulder. A mark is made, and a 22- to 25-gauge, ⅝- to 1-inch needle is carefully directed inferiorly. Rarely is synovial fluid obtained.

The sternoclavicular joint is most easily entered from a point directly anterior to the joint. Caution is necessary to avoid a pneumothorax. The space is fibrocartilaginous, and rarely can fluid be aspirated.

Ankle Joint. For injections of the inhibitors of the present invention in the ankle joints, the patient should be supine and the leg-foot angle at 90 degrees. A mark is made just medical to the tibialis anterior tendon and lateral to the medial malleolus. A 20- to 22-gauge, 1½-inch needle is directed posteriorly and should enter the joint space easily without striking bone.

Subtalar Ankle Joint. Again, the patient is supine and the leg-foot angle at 90 degrees. A mark is made just inferior to the tip of the lateral mallcolus. A 20- to 22-gauge, 1½-inch needle is directed perpendicular to the mark. With this joint the needle may not enter the first time, and another attempt or two may be necessary. Because of this and the associated pain, local anesthesia may be helpful.

Wrist. This is a complex joint, but fortunately most of the intercarpal spaces communicate. A mark is made just distal to the radius and just ulnar to the so-called anatomic snuff box. Usually a 24- to 26-gauge, ⅝ to 1-inch needle is adequate, and the injection is made perpendicular to the mark. If bone is hit, the needle should be pulled back and slightly redirected toward the thumb.

First Carpometacarpal Joint. Degenerative arthritis often involves this joint. Frequently the joint space is quite narrowed, and injections may be difficult and painful. A few simple maneuvers may make the injection fairly easy, however. The thumb is flexed across the palm toward the tip of the fifth finger. A mark is made at the base of the first metacarpal bone away from the border of the snuff box. A 22- to 26-gauge, ⅝ to 1-inch needle is inserted at the mark and directed toward the proximal end of the fourth metacarpal. This approach avoids hitting the radial artery.

Metacarpophalalangeal Joints and Finger Interphalangeal Joints. Synovitis in these joints usually causes the synovium to bulge dorsally, and a 24- to 26-gauge, ½ to ⅝-inch needle can be inserted on the either side just under the extensor tendon mechanism. It is not necessary for the needle to be interposed between the articular surfaces. Some prefer having the fingers slightly flexed when injecting the metacarpophalangeal joints. It is unusual to obtain synovial fluid. When injecting, a mix of the inhibitors of the present invention with a small amount of local anesthetic is preferred.

Metatarsophalangeal Joints and Toe Interphalangeal Joints. The techniques are quite similar to those of the metacarpophalangeal and finger interphalangeal joints, but many prefer to inject more dorsally and laterally to the extensor tendons. Marking the area(s) to be injected is helpful as is gentle traction on the toe of each joint that is injected.

Elbow. A technique preferred by many is to have the elbow flexed at 90 degrees. The joint capsule will bulge if there is inflammation. A mark is made just below the lateral epicondyle of the humerus. A 22-gauge, 1 to 1½-inch is inserted at the mark and directed parallel to the shaft of the radius or directed perpendicular to the skin.

Hip. This is a very difficult joint to inject even when using a fluoroscope as a guide. Rarely is the physician quite sure that the joint has been entered; synovial fluid is rarely obtained. Two approaches can be used, anterior or lateral. A 20-gauge, 3½-inch spinal needle should be used for both approaches.

For the anterior approach, the patient is supine and the extremity fully extended and externally rotated. A mark should be made about 2 to 3 cm below the anterior superior iliac spine and 2 to 3 cm lateral to the femoral pulse. The needle is inserted at a 60 degree angle to the skin and directed posteriorly and medially until bone is hit. The needle is withdrawn slightly, and possibly a drop or two of synovial fluid can be obtained, indicating entry into the joint space.

Many prefer the lateral approach because the needle can “follow” the femoral neck into the joint. The patient is supine, and the hips should be internally rotated—the knees apart and toes touching. A mark is made just anterior to the greater trochanter, and the needle is inserted and directed medially and sightly cephalad toward a point slightly below the middle of the inguinal ligament. One may feel the tip of the needle slide into the joint.

Temporomandibular Joint. For injections, the temporomandibular joint is palpated as a depression just below the zygomatic arch and 1 to 2 cm anterior to the tragus. The depression is more easily palpated by having the patient open and close the mouth. A mark is made and, with the patient's mouth open, a 22-gauge, ½ to 1-inch needle is inserted perpendicular to the skin and directed slightly posteriorly and superiorly.

This invention contemplates embodiments of water soluble silver ion complexes dissolved in standard sterile medical fluid compositions well known in the art that are not highly reactive with biomolecules. Current forms of silver ion compounds are inadequate for parenteral administration to treat internal diseases mediated by matrix metalloproteinases because they are water-insoluble and/or highly reactive biomolecules. This enables embodiments of the present invention capable of parenteral administration according to one of many procedures that are well known in the art.

This invention contemplates preferred embodiments of water soluble silver ion complexes dissolved in standard sterile medical fluid compositions suitable for intrapulmonary administration. The present invention provides a non-invasive means of delivering any type of drug to a patient by the intrapulmonary route. The devices and methodology may require the release of low boiling point propellants in order to aerosolize drug for use with hand-held metered dose inhalers. Alternatively, the devices of the present invention may include other types of hand-held, self-contained, highly portable devices which provide a convenient means of delivering drugs to a patient via the intrapulmonary route.

The inhaled aqueous solution of the present invention may include preservatives or bacteriostatic type compounds. However, the preferred embodiment consists essentially of the pharmaceutically active silver-ion complex and pharmaceutically acceptable vehicle. The aqueous solution may consist essentially of the silver ion complex (i.e. carrier-free) that is freely flowable and can be aerosolized. Aqueous solution comprising silver ion complex may include formulations currently approved for use with nebulizers. Nebulizer formulations are, in general, diluted prior to administration. The aqueous solutions are sterilized and placed in individual containers in a sterile environment. The present invention can provide a means for repeatedly dispensing and delivering the same amount of drug to a patient at each dosing event.

To create an aerosol for either a nebulizer or a medical inhalator device, the formulation is initially forced through the pores of a porous membrane thereby forming streams which are unstable and break up into droplets. The size of the droplets will be affected by factors such as the pore size, temperature, viscosity and the surface tension of the formulation forced through the pores. The streams of liquid are broken into particles having a diameter sufficiently small such that the patient can inhale the particles into the pulmonary tree. Although the particle size will vary depending on factors such as the particular type of formulation being aerosolized, in general, the preferred particle size is in the range of about 0.5 micron to about 12 microns. In order to obtain small particle sizes sufficient to aerosolize a formulation a number of different porous membranes and vibrating devices can be utilized and the present invention is intended to encompass such aerosolizing systems.

The method preferably uses a medical inhalation drug delivery device which is not directly operated by the patient. A preferred device of the invention provides that the drug is delivered automatically upon data received from a monitoring device such as an airflow rate monitoring device. A patient using the medical inhalation device withdraws air from a mouthpiece and the inspiratory rate, and calculated inspiratory volume of the patient is measured simultaneously one or more times in a monitoring event which determines an optimal point in an inhalation cycle for the release of a dose of any desired drug. Inspiratory flow is preferably measured and recorded in one or more monitoring events for a given patient in order to develop an inspiratory flow profile for the patient.

In other embodiments, the water soluble silver ion complexes of the present invention are suspended in a base such as, for example, AQUAPHOR™, polyethylene glycol (PEG) or white petrolatum. This enables embodiments of the present invention capable of topical administration in conjunction with gauze or other absorptive matrices.

In another embodiment, the water-soluble silver ion complexes of the present invention are used either alone or in conjunction with other medicinal (i.e. therapeutic) agents including anti-microbial agents (e.g. antibiotics, antiseptics, etc.), anti-inflammatory agents (e.g. steroids, aspirin, etc.), chelating agents (e.g. EDTA, EGTA, etc.), anti-cancer agents (e.g. taxol, cis-platinum, etc.), and anesthetics (e.g. benzocaine, lidocaine, etc.).

Experimental

In the disclosure which follows, the following abbreviations apply: L (liters); ml (milliliters); μl (microliters); g (grams); mg (milligrams); μg (micrograms); mol (moles); mmol (millimoles); μmol (micromoles); cm (centimeters); mm (millimeters); nm (nanometers); ° C. (degrees Centigrade); MW and M.W. (molecular weight); N (normal); w/w (weight-to-weight); w/v (weight-to-volume); min. (minutes); No. (number); ICP (inductively coupled plasma); CFU (colony forming units); PEG (polyethylene glycol); MHM (Mueller Hinton Medium); ZOI (zone of inhibition); ATCC (American Type Culture Collection, Rockville, Md.); USP (United States Pharmacopeia); NCCLS (National Committee for Clinical Laboratory Standards); NIOSH (National Institute of Safety and Health); Avitar (Avitar, Inc., Canton, Mass.); Aldrich (Milwaukee, Wis.); Avery Dennison, Inc. (Mill Hall, Pa.); BASF (BASF Corp., Chemical Division; Parsippany, N.J.); Belersdorf Inc. (BDF Plaza Norwalk, Conn.); Columbus (Columbus Chemical Industries; Columbus, Wis.); Cook Composites and Polymers (Kansas City, Mo.); Difco (Difco Laboratories, Detroit, Mich.); Hampshire (Hampshire Chemical Co., Lexington, Mass.); Johnson & Johnson Medical, Inc. (Arlington, Tex.); Owen Laboratories (San Antonio, Tex.); Protan (Drammen, Norway); Roundy (Roundy's Inc., Milwaukee, Wis.); Sigma (Sigma Chemical Company, St. Louis, Mo.); SmithKline Beecham (Philadelphia, Pa.); Steriseal (Steriseal Ltd, England); Whatman (Whatman International Ltd., England); WOHL (Wisconsin Occupational Health Laboratory, Madison, Wis.).

The following examples serve to illustrate certain preferred embodiments and aspects of the present invention and are not to be construed as limiting the scope thereof. The examples which follow are categorized into sections as follows: I) Processes To Obtain Silver Thiosulfate Ion Complexes; II) Compositions Containing Silver Thiosulfate Ion Complexes; III) Antimicrobial Activity Of Compositions Containing Silver Thiosulfate Ion Complexes; IV) Matrix Metalloproteinase Inhibitor Activity Of Compositions Containing Silver Thiosulfate Ion Complexes; V) Use Of Silver Thiosulfate Ion Complexes in Medical Devices, and VI) Use Of Silver Thiosulfate Ion Complexes in Combination With Other Medicinal Agents.

I. Processes to Obtain Silver Thiosulfate Ion Complexes

### EXAMPLE 1

### A Process for Making Silver Thiosulfate Ion Complexes Using Silver Chloride When the Ratio of Thiosulfate Ions to Silver Ions is Greater Than 2:1

This example illustrates one embodiment of the process for producing silver thiosulfate ion complexes where the ratio of thiosulfate ions to silver ions is greater than 2:1.

Silver thiosulfate ion complexes were produced by first making a silver chloride precipitate in an aqueous solution (hereafter, “silver chloride precipitate/aqueous solution”). The silver chloride precipitate/aqueous solution was made by mixing 20 ml of a silver nitrate (Aldrich; deionized water as the diluent) solution (1 mmol/ml) with 22 ml of a sodium chloride solution (1 mmol/ml) (Aldrich; deionized water as the diluent) in a 500 ml separatory funnel. To the resulting silver chloride precipitate/aqueous solution was added 60 ml of a sodium thiosulfate (Columbus; deionized water as the diluent) solution (1 mmol/ml). The resulting mixture was agitated by shaking the separatory funnel until all of the silver chloride precipitate was dissolved.

The silver thiosulfate ion complexes produced were separated by adding 200 ml of ethyl alcohol to the container. Upon addition of the ethyl alcohol, the solution became cloudy and separated into two immiscible phases. The two phases were independently collected using a separatory funnel. The weight of the material in the phase containing the silver thiosulfate ion complexes was approximately 17 g. This phase was then treated by adding 70 ml ethyl alcohol and 40 ml of acetone to make the silver thiosulfate ion complexes essentially anhydrous. After sitting overnight, the silver thiosulfate ion complexes were in the form of a pure, white solid material in the bottom of the container. Thereafter, the solvent was decanted and the white solid was dried in an oven (62° C.) and ground to a fine white powder using a mortar and pestle. The weight of the dried silver thiosulfate ion complex powder was 10.03 g.

The silver thiosulfate ion complexes were analyzed for silver, sodium and sulfur using Inductively Coupled Plasma Argon Emission Spectrometry. The analysis, performed by Wisconsin Occupational Health Laboratory (WOHL), included measurement of the amount of silver using a method based on NIOSH SI182. Briefly, a representative portion of the silver thiosulfate ion complexes was weighed and diluted 1/1000 in a dilute nitric acid solution. Thereafter, an aliquot of the sample was analyzed (Jarrel ASH ICP; Franklin, Mass.). This analysis gave the following results expressed as percentages of the air-dried samples: silver 20%, sodium 17% and sulfur 32%.

The results of the analysis suggest that the silver thiosulfate ion complexes were relatively pure and corresponded to the formula: Na₄H[Ag(S₂O₃)₃] (silver: 20.11% (w/w), sodium: 17.13% (w/w), sulfur: 35.75% (w/w)). The calculated yield of the silver thiosulfate ion complexes was 93.7%.

### EXAMPLE 2

### A Process for Making Silver Thiosulfate Ion Complexes Using Silver Chloride When the Ratio of Thiosulfate Ions to Silver Ions is Equal to 2:1

This example illustrates one embodiment of the process for producing silver thiosulfate ion complexes when the ratio of thiosulfate ions to silver ions is equal to 2:1.

Silver thiosulfate ion complexes were produced by first making a silver chloride precipitate in an aqueous solution by mixing 10 ml of a silver nitrate (Aldrich; deionized water as the diluent) solution (1 mmol/ml) with 10 ml of a sodium chloride (Aldrich; deionized water as the diluent) solution (1 mmol/ml) in a 100 ml specimen container. To this silver chloride precipitate/aqueous solution was added 20 ml of a sodium thiosulfate (Columbus; deionized water as the diluent) solution (1 mmol/ml). The resulting mixture was agitated by shaking the container until all of the silver chloride precipitate was dissolved.

Thereafter, the silver thiosulfate ion complexes were separated by adding 50 ml of acetone to the container. Upon addition of the acetone, the solution became cloudy and separated into two immiscible phases. The two phases were collected into individual containers using a pipet. The phase containing the silver thiosulfate ion complexes was treated by adding 50 ml of acetone to make the silver thiosulfate ion complexes essentially anhydrous.

After sitting overnight, the silver thiosulfate ion complexes were in the form of a pure white solid material. Thereafter, the solvent was decanted and the white solid was dried in an oven (62° C.) and ground to a fine white powder using a mortar and pestle. The weight of the dried silver thiosulfate ion complex powder was 3.97 grams.

The resulting silver thiosulfate ion complexes material was analyzed for silver, sodium and sulfur using an Inductively Coupled Plasma (ICP; as described in Example 1). The analysis gave the following results: silver 25%, sodium 17% and sulfur 30%. The results of the analysis indicate that the silver thiosulfate ion complexes were relatively pure corresponding with the following theoretical formula: Na₃[Ag(S₂O₃)₂]2H₂O (silver: 24.7% (w/w), sodium: 15.78% (w/w), sulfur: 29.3% (w/w)). The calculated yield of the silver thiosulfate ion complexes was 90.8%.

### EXAMPLE 3

### A Process for Making Silver Thiosulfate Ion Complexes Using Silver Chloride When the Ratio of Thiosulfate Ions to Silver Ions is Less Than 2:1

This example illustrates a further embodiment of the process for producing silver thiosulfate ion complexes when the ratio of thiosulfate ions to silver ions is less than 2:1.

Silver thiosulfate ion complexes were produced by first making a silver chloride precipitate in an aqueous solution by mixing 10 ml of a silver nitrate (Aldrich; deionized water as the diluent) solution (1 mmol/ml) with 20 ml of a sodium chloride (Aldrich; deionized water as the diluent) solution (1 mmol/ml) in a 100 ml specimen container. To this silver chloride precipitate/aqueous solution was added 15 ml of a sodium thiosulfate (Columbus; deionized water as the diluent) solution (1 mmol/ml). The resulting mixture was agitated by shaking the container until all of the silver chloride precipitate was dissolved.

Thereafter, the silver thiosulfate ion complexes were precipitated from the solution by adding 50 ml of acetone to the container. The precipitated silver thiosulfate ion complexes were in the form of a pure white solid material. The solvent was decanted and the white solid was dried in an oven (62° C.) and ground to a fine white powder using a mortar and pestle.

The silver thiosulfate ion complexes were analyzed for silver, sodium and sulfur using an Inductively Coupled Plasma (ICP; as described in Example I). The analysis gave the following results: silver 32%, sodium 14% and sulfur 29%. The results of the analysis indicate that the silver thiosulfate ion complexes were relatively pure corresponding with the following theoretical formula Na₄[Ag₂(S₂O₃)₃]H₂O (silver: 32.6% (w/w), sodium: 13.9% (w/w), sulfur: 29.0% (w/w)).

### EXAMPLE 4

### A Process for Making Silver Thiosulfate Ion Complexes Using Silver Bromide

This example provides one embodiment that illustrates that silver halides other than chloride may be used to produce silver thiosulfate ion complexes.

Silver thiosulfate ion complexes were produced by first making a silver bromide precipitate in an aqueous solution (hereafter, “silver bromide precipitate/aqueous solution”) by mixing 2 ml of a silver nitrate (Aldrich; deionized water as the diluent) solution (1 mmol/ml) with 2.2 ml of a sodium bromide (Aldrich; deionized water as the diluent) solution (1 mmol/ml) in a 50 ml container. To this silver bromide precipitate/aqueous solution was added 6.0 ml of a sodium thiosulfate (Columbus; deionized water as the diluent) solution (1 mmol/ml). The resulting mixture was agitated by stirring until all of the sodium bromide precipitate was dissolved.

The silver thiosulfate ion complexes were separated by adding 20.0 ml of acetone to the container. Upon addition of the acetone, the solution separated into two immiscible phases. The two phases were collected into individual containers using a pipet. The phase containing the silver thiosulfate ion complexes was treated by adding 7.0 ml ethyl alcohol and 4.0 ml of acetone to make the silver thiosulfate ion complexes anhydrous.

After sitting overnight, the silver thiosulfate ion complexes were in the form of a white solid material at the bottom of the container. The solvent was decanted and the white solid was dried in an oven (62° C.) and ground to a fine white powder using a mortar and pestle. The resulting weight of the dried silver thiosulfate ion complex powder was 0.88 g.

### EXAMPLE 5

### A Process for Making Silver Thiosulfate Ion Complexes without a Phase Separation Procedure

This example illustrates the importance of making silver thiosulfate ion complexes according to, for example, the procedures detailed in Examples I-IV. Silver thiosulfate ion complexes were made by a process which did not use a phase separation procedure where the ratio of thiosulfate ions to silver ions is greater than 2:1.

A silver chloride precipitate was formed in an aqueous solution (hereafter, “silver chloride precipitate/aqueous solution”) by mixing 2 ml of a silver nitrate (Aldrich; deionized water as the diluent) solution (1 mmol/ml) with 2.2 ml of a sodium chloride (Aldrich; deionized water as the diluent) solution (1 mmol/ml) in a 50 ml beaker. To this silver chloride precipitate/aqueous solution was added 6.0 ml of a sodium thiosulfate (Columbus; deionized water as the diluent) solution (1 mmol/ml). The resulting mixture was agitated by stirring until all of the sodium chloride precipitate was dissolved.

The resulting silver thiosulfate ion complexes solution was placed in a convection oven at 62° C. overnight to evaporate the water. The solid material produced had a splotchy tan color with areas which had a deep brown color. The lack of a pure white solid indicates that this process leads to a breakdown or decomposition of silver thiosulfate ion complexes.

II. Compositions Containing Silver Thiosulfate Ion Complexes

### EXAMPLE 6

### Stable Antimicrobial Polyethylene Glycol Base Composition

A silver-based composition was produced having a polyethylene glycol (PEG) base. Specifically, 40 g of a polyethylene glycol (PEG) base (PEG 600:PEG 1000; 0.3:0.7; Aldrich) was melted. Subsequently, 0.47 g of the silver thiosulfate ion complex powder produced according to Example 1 was stirred into the melted PEG base during the cooling process. The stirring was continued until the silver thiosulfate ion complex powder was homogeneously suspended and the cooling process produced a semisolid base comprising a PEG/silver thiosulfate ion complex composition. The amount of silver in this base composition was equivalent to 0.5% silver nitrate.

### EXAMPLE 7

### Stable Antimicrobial AQUAPHOR™ Base Composition

A silver-based composition was produced having an AQUAPHOR™ Cholesterolized Absorbent Eurcerite Ointment base which is a stable, neutral, odorless, anhydrous ointment (Belersdorf Inc). Specifically, 40 g of AQUAPHOR™ was melted. Subsequently, 1.26 g of the silver thiosulfate ion complex powder produced according to Example 1 was stirred into the melted AQUAPHOR™ base during the cooling process. The stirring was continued until the silver thiosulfate ion complex powder was homogeneously suspended and the cooling process produced a semisolid base comprising an AQUAPHOR™/silver thiosulfate ion complex composition. The amount of silver in this silver-based composition was equivalent to 1.0% silver nitrate.

### EXAMPLE 8

### Stable Antimicrobial White Petrolatum Base Composition

A silver-based composition was produced having White Petrolatum USP base. Specifically, 40 g of white petrolatum USP (Roundy's Pure Petroleum Jelly-White Petrolatum USP) was melted. Subsequently, 2.52 g of the silver thiosulfate ion complex powder produced according to Example 1 was stirred into the melted White Petrolatum USP base during the cooling process. The stirring was continued until the silver thiosulfate ion complex powder was homogeneously suspended and the cooling process produced a semisolid base comprising a White Petrolatum USP/silver thiosulfate ion complex composition. The amount of silver in this silver-based composition was equivalent to 2.0% silver nitrate.

III. Antimicrobial Activity of Silver-Ion Complex Compositions

### EXAMPLE 9

### Antimicrobial Activity of Silver Thiosulfate Ion Complexes

In vitro antimicrobial activity was calculated by determining the minimum inhibitory concentration (MIC) for a silver thiosulfate ion complex composition; for example, the silver thiosulfate ion complex produced according to Example 3. The antimicrobial activity of this embodiment was determined by using serial two-fold dilutions ranging from 1.95 to 250 μg/ml using tryptic soy broth (Difco) as a diluent. Each dilution was inoculated with a 0.005 ml aliquot from a 24-hour microbial growth culture having a concentration of approximately 10⁵ to 10⁷ CFU/ml. The dilutions were incubated overnight at 37° C. The MIC was determined by identifying the lowest dilution (expressed as μg/ml) of the silver thiosulfate ion complex that was without evidence of growth (i.e. was not cloudy). The results shown in Table 2 demonstrate that the silver thiosulfate ion complex has antimicrobial activity against both gram (+) and gram (−) microbes (Difco).

TABLE 2
Silver Thiosulfate Ion Antimicrobial Activity Against Gram Negative And
Positive Microbes

| Isolate | ATCC Accession No. | Complexes (μg/ml) |
|---|---|---|
| S. aureus | 25923 | <1.95 |
| S. epidermidis | 12228 | <1.95 |
| E. coli | 25922 | <1.95 |
| P. aeruginosa | 27853 | <1.95 |

### EXAMPLE 10

### Zone-of-Inhibition Antimicrobial Assay

This example provides one embodiment of a method for evaluating antimicrobial activity. The antimicrobial activity of the silver-ion complex compositions produced according to Examples 6, 7, and 8 were evaluated using a zone-of-inhibition (ZOI) protocol. First, 1 cm-diameter paper discs (Whatman Filter Paper, Quantitative 1) were coated with a thin layer of the above silver-ion complex compositions. These coated paper discs were placed on 24 hour growth lawns of S. aureus (ATCC 25923) plated on Mueller Hinton Medium (MHM; Difco). After incubation at 36° C. for 18 hours, antimicrobial activity was determined by measuring the width (in mm) of the ZOI ring surrounding the paper disc, identified as a clear area on the culture medium surface, from the edge of the paper disc to the point where microbial growth resumes. Table 3 compares ZOI results for each composition of measurements taken Day 1 (i.e., after 18 hours of incubation) and again one month later.

TABLE 3
Antimicrobial Activity Of Silver-Based Compositions Against
Staphylococcus aureus: Zone of Inhibition

| Sample | Day 1 | 1 Month |
|---|---|---|
| PEG Composition | 13.5 mm | 14.0 mm |
| (Example 6) | | |
| AQUAPHOR ™ | 10.0 mm | 13.0 mm |
| Composition (Example 7) | | |
| White Petrolatum | 10.0 mm | 10.5 mm |
| Composition (Example 8) | | |

The results of this study demonstrate that these exemplary silver-ion complex compositions have significant antimicrobial activity that are stable for at least one month. That is to say, the size of the zone of growth inhibition was essentially unchanged over the one month period. Therefore, as discussed above, it may be expected that these compositions also function as matrix metalloproteinase inhibitors.

### EXAMPLE 11

### Matrix Metalloproteinase Inhibition by Silver Thiosulfate Ion Complexes

This example outlines a method to determine the inhibition of metalloproteinase activity by silver thiosulfate ion complexes. Specifically, this example provides that a substrate is placed in contact with a protein that degrades at least a portion of the substrate. In this example, the substrate is collagen and the protein is collagenase. An aqueous silver thiosulfate ion complex is added in vitro to the solution containing collagen and collagenase, and the resultant collagenase activity is colorimetrically measured.

Collagenase activity is measurable by methods known in the art. (e.g., U.S. Pat. No. 5,686,422 To Gray et al., Synthetic inhibitors of mammalian collagenase). This example explains how a spectrophotometric method is utilized to determine collagenase activity (Lindy, S. et al., European J. Biochem. 156: 1-4 (1986)). Acid-soluble calf skin collagen (0.25 mg/ml, approximately 0.8 M) is incubated at 35° C. for 1 hr with pig synovial collagenase (0.04 μg protein) in 0.05 M tris-HCL, 0.2M NaCl, 0.25M glucose, 5 mM CaCl₂, 10% dimethyl sulfoxide, pH 7.6 in a reaction volume of 200 μL having a temperature of 37° C. and an enzyme concentration of 1.2 μg protein/ml. Proposed metalloproteinase inhibitors, such as the silver thiosulfate ion complex aqueous powder produced in accordance with either Example 1 or Example 3, are dissolved in 1 mM acetic acid/ethanol stock solutions. The reaction progress is monitored for 6-10 minutes by following increases in absorbance at 227 nm that accompanies the denaturation of collagen fragments. (Ellman, G. L., Arch. Biochem. Biophys. 82: 70-77 (1959). Initial rates of collagen degradation are determinable from the linear portion of the time-course curves.

This method reliably determines the extent of metalloproteinase inhibition by silver thiosulfate ion complex solutions. Due to the direct nature of this inhibition, it is expected that similar in vivo inhibition of related metalloproteinase enzymes will result during either topical, intra-articular or parental administration of silver thiosulfate ion complex solutions to a subject.

V. Use of Silver Thiosulfate Ion Complexes in Medical Devices

### EXAMPLE 12

### Foam Dressings Containing Silver Thiosulfate Ion Complexes

This example illustrates the production of a polymer foam matrix dressing comprising a silver thiosulfate ion complex. Specifically, the silver thiosulfate ion complexes were incorporated into the foam during the manufacturing of the polymer matrix. For example, a foam dressing was produced by first dissolving 0.54 g of the silver thiosulfate ion complex powder produced according to Example 1 in 150 ml of a 0.5% Pluronic L-62 (BASF) aqueous solution. This solution was then mixed with 140 g of a polyurethane pre-polymer (Hypol 2002, Hampshire) in a 1-liter disposable plastic beaker. The resulting mixture instantly began to react to form a foam. After 10 minutes the silver thiosulfate containing foam was removed from its container and sliced to produce individual foam dressings (approximately 7.5 cm in diameter). The slices of foam dressings were dried at 50° C. in a dark convection oven. These silver thiosulfate foam dressings were light stable and antimicrobially active according to the ZOI test. These foam dressings can be used for a large variety of medical applications, including as an antimicrobial matrix metalloproteinase inhibitor absorptive foam dressings.

### EXAMPLE 13

### Foam Dressing Containing Silver Thiosulfate Ion Complexes

This example illustrates the application of silver thiosulfate ion complexes onto a foam polymer matrix medical device following the matrix's manufacture.

Foam dressing squares (HYDRASORB™ Sponge Foam Dressing: 10 cm×10 cm; Avitar) were submerged in a 0.1 g/liter silver thiosulfate ion complex aqueous solution produced in accordance with Example 3. The foam dressing squares were removed and dried at 50° C. in a convection oven. These silver thiosulfate ion complex-containing foam dressings were light stable and antimicrobially active according to the ZOI test. These foam dressings can be used for a large variety of medical applications, including as an antimicrobial matrix metalloproteinase inhibitor absorptive foam dressings.

### EXAMPLE 14

### Hydrocolloid Dressing Containing Silver Thiosulfate Ion Complexes

This example illustrates the incorporation of the silver thiosulfate ion complexes to prepare a medical device having a hydrocolloid absorbent polymer matrix. In this example, the complexes were incorporated into the matrix during the manufacturing of the polymer matrix.

A hydrocolloid dressing containing silver thiosulfate ion complexes was produced by first thoroughly mixing 0.157 g of silver thiosulfate ion complex powder (mesh>100) produced in accordance with Example 1 with 10.0 g of sodium carboxymethyl cellulose (Aldrich). Thereafter, 4 g of this silver thiosulfate ion complex/carboxymethyl cellulose composition was mixed thoroughly with 4 g of a polyurethane prepolymer (Aquapol 035-0031, Cook Composites and Polymers). This polyurethane prepolymer mixture was then pressed between a polyurethane film and a silicone-treated hydrocolloid matrix and allowed to cure for 24 hours. The resulting silver thiosulfate ion complex-containing hydrocolloid dressing was photostable and antimicrobially active. This type of dressing is useful on exudating, malodorous wounds.

### EXAMPLE 15

### Hydrocolloid Dressing Containing Silver Thiosulfate Ion Complexes

This example illustrates an alternative method for Example 14 to produce a silver thiosulfate ion complex containing hydrocolloid absorbent polymer matrix medical device.

The hydrocolloid dressing was produced by first dissolving 0.157 g of a silver thiosulfate ion complex powder (mesh>100) produced in accordance with Example 1 in 10.0 ml of water. This aqueous solution was absorbed into 100 g of sodium carboxymethyl cellulose (Aldrich, Milwaukee, Wis.). The silver thiosulfate ion complex/sodium carboxymethyl cellulose composition was allowed to dry at room temperature. Thereafter, 4 g of the dried composition was mixed thoroughly with 4 g of a polyurethane prepolymer (Aquapol 035-0031, Cook Composites and Polymers). This mixture was then pressed between a polyurethane film and a silicone-treated liner and allowed to cure for 24 hours.

The resulting silver thiosulfate ion complex-containing hydrocolloid dressing was photostable and antimicrobially active. This type of dressing is useful on exudating, malodorous wounds.

### EXAMPLE 16

### Adhesive Films Containing Silver Thiosulfate Ion Complexes

This example illustrates the use of silver thiosulfate ion complexes to produce adhesive films; specifically, a pressure sensitive adhesive (PSA) film. Adhesive films are, among other things, especially useful in covering painful abrasive-type skin wounds and partial skin graft sites.

First, an adhesive solution consisting of 45 g of a proprietary medical grade acrylic based latex (containing 58% solids; Avery Dennison, Inc.) and 5 g polyethylene glycol (600 MW; Aldrich) was prepared. Then, 0.25 g of the silver thiosulfate ion complex powder produced in accordance with Example 1 was mixed with the adhesive solution, thus forming an adhesive mixture. This adhesive mixture, when coated on a surface and air-dried, produces a tacky, adhesive film.

The adhesive film is photostable and antimicrobially active. This adhesive film can be laminated to dressing backing materials to produce dressings which are antimicrobially active. Dressings with the silver thiosulfate ion complex-containing PSA are especially useful in covering painful abrasive-type skin wounds and partial skin graft sites.

### EXAMPLE 17

### Alginate Materials Containing Silver Thiosulfate Ion Complexes

This example illustrates the incorporation of silver thiosulfate ion complexes into a medical device comprising a non-adherent alginate material. Briefly, this method involves the use of a silver thiosulfate ion complex/calcium chloride bath that crosslinks alginate fibers and incorporates the silver thiosulfate ion complexes into the alginate fibers during their formation.

Alginate fibers were made by using a syringe to inject a 5% sodium alginate solution (Pronova LV M Sodium Alginate, Protan) into a bath containing a 10% calcium chloride solution (Aldrich, deionized water as diluent) and 0.1 g/liter silver thiosulfate ion complex powder produced in accordance with Example 3. The alginate solution immediately formed water-insoluble alginate fibers upon contact with the calcium chloride/silver thiosulfate ion complex solution. The water-insoluble silver thiosulfate ion complex-containing fibers were then pulled from the bath and allowed to dry at 50° C.

The resulting fibers are photostable and antimicrobially active. These fibers can be used to make antimicrobial alginate dressings and tamponades. Alginate materials containing silver thiosulfate ion complexes are especially useful in covering painful abrasive-type skin wounds and wound ulcers as well as for filling in deep wounds and cavities.

### EXAMPLE 18

### Alginate Materials Containing Silver Thiosulfate Ion Complexes

This example illustrates an alternative method to Example 17 to produce a medical device comprising non-adherent alginate material and silver thiosulfate ion complexes. This method does not utilize a calcium chloride bath.

First, an aqueous solution was prepared containing 0.1 g/liter of a silver thiosulfate ion complex powder produced in accordance with Example 3. The resulting silver thiosulfate ion complex aqueous solution was then sprayed onto a 9.5 cm×9.5 cm alginate dressing square (Steriseal Sorbsan Surgical Dressing, Steriseal). Alternatively, the alginate dressing may be dipped into the aqueous silver thiosulfate ion complex solution. The alginate fibers of the dressing are then allowed to absorb the solution. Thereafter, the treated alginate dressing was allowed to dry at room temperature.

The alginate dressing was light stable and antimicrobially active and are especially useful for malodorous wounds as well as for covering painful abrasive-type skin wounds and wound ulcers.

VI. Silver Thiosulfate Ion Complexes Combined with Other Medicinal Agents

### EXAMPLE 19

### Pharmaceutical Composition Combining EDTA with Silver Thiosulfate Ion Complexes

This example suggests one embodiment for a method to produce a pharmaceutical composition combining EDTA with silver thiosulfate ion complexes.

An antimicrobial matrix metalloproteinase inhibitor pharmaceutical composition may be produced where a silver thiosulfate ion complex powder is combined with one or more compounds comprising ethylenediamine tetraacetic acid (EDTA; Sigma). Specifically, 0.25 g of the silver thiosulfate ion complex produced in accordance with Example 3 is added to 24.50 g of a PEG base composition that is produced by melting together 40% PEG (3450 MW) and 60% PEG (600 MW). The first melting results in a pharmaceutical composition into which the silver thiosulfate ion complexes and EDTA are stirred. This final pharmaceutical composition is stirred continually until cooling results in resolidification. The resulting pharmaceutical composition is expected to have broad spectrum topical antimicrobial on chronic wounds that are caused by matrix metalloproteinase activity.

From the above Examples, it should be evident that the present invention provides for producing silver ion-based matrix metalloproteinase inhibitor compositions using such composition in the treatment of infectious disease states and/or conditions associated with excessive tissue destruction. It should be understood that the present invention is not limited to the embodiments shown. In light of the foregoing disclosure, it will be apparent to those skilled in the art that substitutions, alterations, and modifications are possible in the practice of this invention without departing from the spirit or scope thereof.

We claim:

1. A method, comprising:
a) providing:
i) a subject, exhibiting at least one symptom associated with a metalloproteinase-associated disease; and
ii) an aqueous solution comprising an effective amount of a water soluble silver-ion complex; and
b) administering said solution to said subject under conditions such that at least one symptom is reduced.

2. The method of claim 1, wherein said complex directly inhibits matrix metalloproteinase activity.

3. The method of claim 1, wherein said complex comprises a non-toxic ligand.

4. The method of claim 1, wherein said symptom of comprises tissue destruction.

5. The method of claim 4, wherein said tissue destruction is internal.

6. The method of claim 1, wherein said administering is selected from the group consisting of intra-articular injection, parenteral injection and topical application.

7. The method of claim 1, wherein said solution further comprises medicinal agents selected from the group consisting of anti-microbials, steroid compounds, non-steroidal anti-inflammatory agents, metal chelators, anti-cancer agents, and anesthetics.

8. The method of claim 1, wherein said subject is a human.

9. The method of claim 1, wherein said metalloproteinase-associated disease is selected from the group consisting of osteoarthritis, rheumatoid arthritis, and septic arthritis.

10. The method of claim 1, wherein said silver-ion complex comprises silver thiosulfate.

11. The method of claim 12, wherein said complex is carrier-free.

12. A method, comprising:
a) providing:
i) a subject, exhibiting at least one symptom associated with rheumatoid arthritis; and
ii) an aqueous solution comprising an effective amount of a water soluble silver-ion complex; and
b) administering said solution to said subject under conditions such that at least one symptom is reduced.

13. The method of claim 12, wherein said complex directly inhibits matrix metalloproteinase activity.

14. The method of claim 12, wherein said administering is selected from the group consisting of intra-articular injection, parenteral injection and topical application.

15. The method of claim 12, wherein said solution further comprises medicinal agents selected from the group consisting of anti-microbials, steroid compounds, non-steroidal anti-inflammatory agents, metal chelators, anti-cancer agents, and anesthetics.

16. The method of claim 12, wherein said subject is a human.

17. The method of claim 12, wherein said silver-ion complex comprises silver thiosulfate.

18. The method of claim 17, wherein said complex is carrier-free.

19. A method, comprising:
a) providing:
i) a subject, exhibiting at least one symptom associated with osteoarthritis;
ii) an aqueous solution comprising an effective amount of a water soluble silver-ion complex; and
b) administering said solution to said subject under conditions such that at least one symptom is reduced.

20. The method of claim 19, wherein said complex directly inhibits matrix metalloproteinase activity.

21. The method of claim 19, wherein said administering is selected from the group consisting of intra-articular injection, parenteral injection and topical application.

22. The method of claim 19, wherein said solution further comprises medicinal agents selected from the group consisting of anti-microbials, steroid compounds, non-steroidal anti-inflammatory agents, metal chelators, anti-cancer agents, and anesthetics.

23. The method of claim 19, wherein said subject is a human.

24. The method of claim 19, wherein said silver-ion complex comprises silver thiosulfate.

25. The method of claim 24, wherein said complex is carrier-free.

26. A method, comprising:
a) providing:
i) a subject, wherein said subject has a wound;
ii) an aqueous solution comprising an effective amount of a water soluble silver-ion complex; and
b) administering said solution to said subject under conditions such that the severity of said wound is reduced.

27. The method of claim 26, wherein said administering is topical administration.

28. The method of claim 27, wherein said topical administration comprises an absorptive matrix.

29. The method of claim 26, wherein said complex directly inhibits matrix metalloproteinase activity.

30. The method of claim 26, wherein solution further comprises medicinal agents selected from the group consisting of anti-microbials, steroid compounds, non-steroidal anti-inflammatory agents, metal chelators, anti-cancer agents, and anesthetics.

31. The method of claim 26, wherein said subject is a human.

32. The method of claim 26, wherein said silver-ion complex comprises silver thiosulfate.

33. The method of claim 32, wherein said complex is carrier-free.
